(19) United States
(12) Patent Application Publication
(10) Pub. No.: US 2026/0294917 A1
(43) Pub. Date: Oct. 1, 2026

(54) INGESTIBLE FORMULATIONS COMPRISING METHYLLIBERINE AND ONE OR MORE SUGARS AND THEIR USE TO TREAT THE ADVERSE EFFECTS OF ALCOHOL CONSUMPTION

(71) Applicant: LUCID PSYCHECEUTICALS INC., Toronto (CA)

(72) Inventors: Lakshmi Premakanth KOTRA, Toronto (CA); Ashwini JOSHI, Toronto (CA); Ewa WASILEWSKI, Toronto (CA); Andrzej CHRUSCINSKI, Toronto (CA)

(21) Appl. No.: 19/477,238

(22) PCT Filed: Apr. 23, 2024
(86) PCT No.: PCT/IB2024/053952

§ 371 Date: Oct. 21, 2025

Related U.S. Application Data
(60) Provisional application No. 63/497,772, filed on Apr. 24, 2023.

(51) Int. Cl. A61K 31/522; A61K 9/00; A61K 31/198; A61K 31/352; A61K 31/357; A61K 31/455; A61K 31/4748; A61K 31/51; A61K 31/525; A61K 31/675; A61K 31/7004; A61K 31/714; A61K 33/00; A61K 33/06; A61K 33/42; A61K 36/28; A61K 36/87; A61K 47/12; A61K 47/24; A61P 3/00; A61P 25/32

(52) U.S. Cl. CPC A61K 31/522; A61K 9/0095; A61K 31/198; A61K 31/352; A61K 31/357; A61K 31/455; A61K 31/4748; A61K 31/51; A61K 31/525; A61K 31/675; A61K 31/7004; A61K 31/714; A61K 33/00; A61K 33/06; A61K 33/42; A61K 36/28; A61K 36/87; A61K 47/12; A61K 47/24; A61P 3/00; A61P 25/32

(57) ABSTRACT

Ingestible formulations that include one or more sugars, such as fructose, and methylliberine. The ingestible formulations may additionally include dihydromyricetin (DHM) and/or Huperzine A. The ingestible formulations may additionally include one or more of cognitive enhancers, neurostimulants, cofactors, and general nutrition support. The ingestible formulations may be used to accelerate aldehyde or alcohol metabolism in a subject, reduce or reverse one or more negative effects of alcohol on motor or cognition performance in a subject, accelerate the reduction of blood-alcohol concentration (BAC) in a subject, and/or aid in the metabolism of an alcohol-containing food or beverage by a subject.

### CROSS REFERENCE TO RELATED APPLICATION

This application is the U.S. national stage of International Patent Application No. PCT/IB2024/053952, filed Apr. 23, 2024, which claims the benefit of U.S. Provisional Application No. 63/497,772 filed on Apr. 24, 2023, the entire contents of which is incorporated by reference herein.

### FIELD

This specification relates to ingestible formulations used to remedy one or more effects of excessive alcohol consumption.

### BACKGROUND

The following paragraphs are not an admission that anything discussed in them is prior art or part of the knowledge of persons skilled in the art.

Consumption of ethanol, also referred to herein as “alcohol”, or ethanol-containing products or products that generate ethanol after consumption, and especially excessive consumption of such products, may be accompanied by a variety of symptoms, such as headaches, inebriation, loss of motor control, nausea, dizziness, insomnia, dehydration and fatigue. Generally, alcohol products consumption leads to buzz in the head, feeling of happiness or high, and sometimes mild sedation. One or more symptoms may be related to the metabolism of the ingested alcohol into alcohol metabolism byproducts, such as aldehydes and aldehyde-based adducts.

### SUMMARY

According to various aspects of the disclosure, a first aspect can be described as ingestible formulations comprising one or more sugars selected from the group consisting of fructose, high fructose corn syrup (HFCS), sucrose (for example, table sugar) and lactose (for example, milk sugar); and methylliberine. In some instances, the methylliberine is provided by coffee beans, tea, cola nuts, guarana, cocoa, and/or yerba mate. In some instances, the methylliberine is provided as an isolate from coffee beans, tea, cola nuts, guarana, cocoa, and/or yerba mate.

According to various aspects of the disclosure, a second aspect can be described as ingestible formulations according to the first aspect, wherein the one or more sugars is fructose.

According to various aspects of the disclosure, a third aspect can be described as ingestible formulations according to the first or second aspect, wherein the ingestible formulations comprise about 1 milligrams (mg) to about 150 mg of the methylliberine, and about 2 grams (g) to about 100 g of the fructose.

According to various aspects of the disclosure, a fourth aspect can be described as ingestible formulations according to any one of the first through third aspects, wherein the ingestible formulations further comprise dihydromyricetin (DHM). In some instances, the DHM is provided by kale, oranges, or a vine tea extract.

According to various aspects of the disclosure, a fifth aspect can be described as ingestible formulations according to the fourth aspect, wherein the ingestible formulations comprise about 50 mg to about 1000 mg of DHM.

According to various aspects of the disclosure, a sixth aspect can be described as ingestible formulations according to any one of the first through fifth aspects, wherein the ingestible formulations further comprise an extract of Huperzia serrata (containing 1 w/w % Huperzine A).

According to various aspects of the disclosure, a seventh aspect can be described as ingestible formulations according to the sixth aspect, wherein the ingestible formulations comprise about 0.1 micrograms (μg) to about 200 μg of the Huperzine A.

According to various aspects of the disclosure, an eighth aspect can be described as ingestible formulations according to any one of the first through seventh aspects, wherein the ingestible formulations further comprise caffeine. In some instances, the ingestible formulations have up to 400 mg of caffeine.

According to various aspects of the disclosure, a ninth aspect can be described as ingestible formulations according to any one of the first through eighth aspects, wherein the ingestible formulations further comprise, methylcobalamin, pyridoxal-5-phosphate (P5P), pyridoxine HCl, thiamine, riboflavin, niacin, nicotinamide, quercetin, L-phenylalanine, calcium citrate, magnesium citrate, sodium citrate, and potassium chloride or monopotassium phosphate.

According to various aspects of the disclosure, a tenth aspect can be described as ingestible formulations according to any one of the first through ninth aspects, wherein the ingestible formulations further comprise one or more about 5 mg to about 100 mg of niacin, nicotinamide, or a combination of niacin and nicotinamide; about 50 mg to about 1,200 mg of quercetin, about 250 mg to about 1,000 mg of L-phenylalanine, about 0.1 mg to about 5.0 mg of methylcobalamin; about 5 mg to about 100 mg of pyridoxal 5-phosphate, pyridoxine HCl, or a combination of pyridoxal 5-phosphate and pyridoxine HCl; about 0.5 mg to about 3 mg of thiamine; about 2.5 mg to about 10 mg of riboflavin; about 100 mg to about 400 mg of calcium citrate, about 100 mg to about 400 mg of magnesium citrate, about 100 mg to about 400 mg of sodium citrate, and about 100 mg to about 400 mg of potassium chloride or monopotassium phosphate.

According to various aspects of the disclosure, an eleventh aspect can be described as ingestible formulations according to any one of the first through tenth aspects, wherein the ingestible formulations further comprise milk thistle, an extract of milk thistle, or silymarin.

According to various aspects of the disclosure, a twelfth aspect can be described as ingestible formulations according to the eleventh aspect, wherein the ingestible formulations comprise about 30 mg to about 600 mg of the extract of milk thistle or silymarin.

According to various aspects of the disclosure, a thirteenth aspect can be described as ingestible formulations according to any one of the first through twelfth aspects, wherein the ingestible formulations further comprise a preservative, and a flavouring agent.

According to various aspects of the disclosure, a fourteenth aspect can be described as ingestible formulations according to the thirteenth aspect, wherein the ingestible formulations further comprise a pH adjustment component and water.

According to various aspects of the disclosure, a fifteenth aspect can be described as ingestible formulations according to the thirteenth or fourteenth aspect, wherein the preservative is potassium sorbate, sodium benzoate, monopotassium phosphate, citric acid, or any combination thereof.

According to various aspects of the disclosure, a sixteenth aspect can be described as ingestible formulations according to any one of the first through twelfth aspects, wherein the ingestible formulations preserved by a preservation method such as pasteurization.

According to various aspects of the disclosure, a seventeenth aspect can be described as ingestible formulations according to the sixteenth aspect, wherein the ingestible formulations further comprise a flavouring agent.

According to various aspects of the disclosure, an eighteenth aspect can be described as ingestible formulations according to the sixteenth or seventeenth aspect, wherein the ingestible formulations further comprise a pH adjustment component and water.

According to various aspects of the disclosure, a nineteenth aspect can be described as ingestible formulations according to any one of the first through eighteenth aspects, wherein the ingestible formulations comprise one or more of B12 (methylcobalamin), B6 (pyridoxal 5-phosphate or pyridoxine hydrochloride or both), B1 (thiamine or thiamine HCl), B2 (riboflavin), magnesium citrate, sodium citrate, potassium chloride or monopotassium phosphate, potassium sorbate, sodium benzoate, citric acid, lecithin, sucralose, quercetin (or equivalent amount of SunActive® Iso Q or other quercetin-delivering compounds), milk thistle, L-phenylalanine, optionally organic green tea extract (caffeine), optionally niacin and/or NAT.

According to various aspects of the disclosure, a twentieth aspect can be described as ingestible formulations according to any one of the first through nineteenth aspects, wherein the ingestible formulations comprise, consist essentially of, or consist of fructose, B12 (methylcobalamin), B6 (pyridoxal 5-phosphate), B1 (thiamine), B2 (riboflavin), magnesium citrate, sodium citrate, potassium chloride (or monopotassium phosphate), potassium sorbate, sodium benzoate, citric acid, lecithin, sucralose, vine tea extract (DHM), methylliberine (or a methylliberine containing compound), quercetin (or equivalent amount of SunActive® Iso Q or other quercetin delivering compounds), milk thistle, L-phenylalanine, Huperzine A, optionally Organic green tea extract (caffeine), optionally niacin and/or N-acetyl tyrosine (NAT).

According to various aspects of the disclosure, a twenty-first aspect can be described as ingestible formulations according to the twentieth aspect, wherein the ingestible formulations comprise, consist essentially of, or consist of 2-100 g fructose, 10-400 mcg B12 (methylcobalamin), 1-30 mg, B6 (pyridoxal 5-phosphate or an equivalent salt), 0.5-5 mg B1 (thiamine or an equivalent salt), 0.5-15 mg B2 (riboflavin), 10-400 mg magnesium citrate, 10-400 mg sodium citrate, 10-400 mg potassium chloride or monopotassium phosphate, 0.005%-0.1% w/v potassium sorbate, 0.01%-0.1% w/v sodium benzoate, 0.05%-0.5% w/v citric acid, 0.05%-1% w/v lecithin, 0.02%-0.1% w/v sucralose, 50 mg-1000 mg DHM (preferably from vine tea extract or Hovenia dulcis extract, etc.), 1 mg-150 mg methylliberine (or an equivalent amount of methylliberine containing compounds), 250 mg-1,200 mg Quercetin (or an equivalent amount of SunActive® Iso Q or other products containing quercetin), 30 mg-600 mg milk thistle, 250 mg-1,000 mg L-phenylalanine, 1 mcg-200 mcg Huperzine A, 0 mg-400 mg organic green tea extract (caffeine), 0-100 mg Niacin, and 0-1,500 mg NAT.

According to various aspects of the disclosure, a twenty-second aspect can be described as ingestible formulations according to the twentieth aspect, wherein the ingestible formulations comprise, consist essentially of, or consist of 6 g fructose, 178 mcg B12 (methylcobalamin), 11 mg B6 (pyridoxal 5-phosphate or an equivalent salt), 1.4 mg B1 (thiamine or an equivalent salt), 5 mg B2 (riboflavin), 200 mg magnesium citrate, 200 mg sodium citrate, 200 mg potassium chloride or monopotassium phosphate, 0.03% w/v potassium sorbate, 0.03% w/v sodium benzoate, 0.15% w/v citric acid, 0.25% w/v Lecithin, 0.02% w/v Sucralose, 150 mg DHM (from Vine tea extract), 48 mg methylliberine (or an equivalent amount of methylliberine containing compounds), 500 mg quercetin (or an equivalent amount of SunActive® Iso Q or other products containing quercetin), 60 mg milk thistle, 500 mg L-phenylalanine, 125 mcg Huperzine A, 150 mg organic green tea extract (caffeine), and 10 mg Niacin.

According to various aspects of the disclosure, a twenty-third aspect can be described as ingestible formulations according to the twentieth aspect, wherein the ingestible formulations comprise, consist essentially of, or consist of 6 g fructose, 178 mcg B12 (methylcobalamin), 11 mg B6 (pyridoxal 5-phosphate or an equivalent salt), 1.4 mg B1 (thiamine or an equivalent salt), 5 mg B2 (riboflavin), 200 mg magnesium citrate, 200 mg sodium citrate, 200 mg potassium chloride or monopotassium phosphate, 0.03% w/v potassium sorbate, 0.03% w/v sodium benzoate, 0.15% w/v citric acid, 0.25% w/v lecithin, 0.02% w/v sucralose, 600 mg DHM (from Vine tea extract), 60 mg methylliberine (or equivalent amount of methylliberine containing compounds), 500 mg quercetin (or equivalent amount of SunActive® Iso Q or other products containing quercetin), 60 mg milk thistle, 500 mg L-phenylalanine, and 150 mcg Huperzia serrata extract comprising 1 w/w % Huperzine A.

According to various aspects of the disclosure, a twenty-fourth aspect can be described as ingestible formulations according to the twentieth aspect, wherein the ingestible formulations comprise, consist essentially of, or consist of 6 g fructose, 178 mcg B12 (methylcobalamin), 11 mg B6 (pyridoxal 5-phosphate or an equivalent salt), 1.4 mg B1 (thiamine or an equivalent salt), 5 mg B2 (riboflavin), 200 mg magnesium citrate, 200 mg sodium citrate, 200 mg potassium chloride or monopotassium phosphate, 0.03% w/v potassium sorbate, 0.03% w/v sodium benzoate, 0.15% w/v citric acid, 0.25% w/v lecithin, 0.02% w/v sucralose, 400 mg DHM (from Vine tea extract), 48 mg methylliberine (or equivalent amount of methylliberine containing compounds), 500 mg quercetin (or equivalent amount of SunActive® Iso Q or other products containing quercetin), 180 mg milk thistle, 500 mg L-phenylalanine, and 1.50 mcg Huperzine A, 150 mg organic green tea extract (caffeine), 10 mg Niacin and optionally 500 mg NAT.

According to various aspects of the disclosure, a twenty-fifth aspect can be described as ingestible formulations according to any one of the first through eighteenth aspects, wherein the ingestible formulations comprise fructose, methylcobalamin, pyridoxine hydrochloride, thiamine hydrochloride, riboflavin, niacin, magnesium citrate, sodium citrate, monopotassium phosphate, vine tea extract, methylliberine (or a methylliberine-containing compound or composition), quercetin (or a quercetin-containing compound or composition), milk thistle or an extract thereof, L-phenylalanine, Huperzia serrata (Huperzine A), and green tea extract.

According to various aspects of the disclosure, a twenty-sixth aspect can be described as ingestible formulations according to the twenty-fifth aspect, wherein the ingestible formulations comprise, consist essentially of or consist of 6 g fructose, 178 mcg methylcobalamin, 10 mg pyridoxine hydrochloride, 1.4 mg thiamine hydrochloride, 5 mg riboflavin, 10 mg niacin, 200 mg magnesium citrate, 200 mg sodium citrate, 200 mg monopotassium phosphate, 600 mg of DHM in the form of Vine tea extract, 60 mg methylliberine (or a methylliberine-containing compound or composition), 181 mg quercetin (or a quercetin-containing compound or composition), 60 mg milk thistle or an extract thereof, 500 mg L-phenylalanine, 150 mcg Huperzia serrata extract comprising 1 w/w % Huperzine A, and 200 mg caffeine in the form of green tea extract.

According to various aspects of the disclosure, a twenty-seventh aspect can be described as ingestible formulations according to the twenty-fifth or twenty sixth aspect, wherein the ingestible formulations further comprise one or more of sucralose, xanthan gum and citric acid.

According to various aspects of the disclosure, a twenty-seventh aspect can be described as ingestible formulations according to and one of the twenty-fifth through twenty seventh aspects, wherein the ingestible formulations further comprise comprising 0.01-0.03% w/w sucralose, and/or 0.04-0.06% w/w xanthan gum, and/or 0.2-0.5% w/w citric acid.

According to various aspects of the disclosure, a twenty-eighth aspect can be described as ingestible formulations according to any one of the first through twenty-seventh aspects, wherein the ingestible formulations are in the form of a beverage.

According to various aspects of the disclosure, a twenty-ninth aspect can be described as ingestible formulations according to any one of the first through twenty-seventh aspects, wherein the ingestible formulations are in the form of a solid dosage form that is dissolvable in water, a juice, or another ingestible fluid.

According to various aspects of the disclosure, a thirtieth aspect can be described as ingestible formulations according to any one of the first through twenty-seventh aspects, wherein the ingestible formulations are in the form of a powder that is dissolvable or dispersible in water, a juice, or another ingestible fluid.

According to various aspects of the disclosure, a thirty-first aspect can be described as ingestible formulations according to any one of the first through thirtieth aspects, wherein the ingestible formulations are for administration to a subject after the subject has consumed alcohol, and administration is for the purpose of enhancing mental alertness of the subject, promoting acceleration of alcohol metabolism in the subject, and providing general nutritional support to the subject to alleviate acute effects of alcohol consumption.

According to various aspects of the disclosure, a thirty-second aspect can be described as ingestible formulations prepared by a process comprising mixing methylliberine and one or more sugars selected from the group consisting of fructose, high fructose corn syrup (HFCS), sucrose (for example, table sugar) and lactose (for example, milk sugar) in a solution comprising water in amounts to result in a formulation that comprises from about 0.12 mg to about 3.3 mg of the methylliberine per mL of the formulation; and from about 11 mg to about 125 mg of the one or more sugars per mL of the formulation.

According to various aspects of the disclosure, a thirty-third aspect can be described as ingestible formulations according to the thirty-second aspect, wherein the one or more sugars is fructose.

According to various aspects of the disclosure, a thirty-fourth aspect can be described as ingestible formulations according to the thirty-second or thirty-third aspect, wherein the process further comprises (a) mixing dihydromyricetin (DHM) in the solution, preferably in an amount to result in the formulation comprising from about 1.05 mg to about 3.0 mg of the DHM per mL of the formulation; or (b) mixing Huperzine A in the solution, preferably in an amount to result in the formulation comprising from about 0.26 μg to about 6.6 μg of the Huperzine A per 355 mL of the formulation; or (c) mixing caffeine in the solution, preferably in an amount to result in the formulation comprising from 0.28 mg to about 0.66 mg of the caffeine per mL of the formulation; or (d) any combination of (a), (b) and (c).

According to various aspects of the disclosure, a thirty-fifth aspect can be described as ingestible formulations according to the thirty-second or thirty-third aspect, wherein the dihydromyricetin (DHM) is mixed in the solution in an amount to result in the formulation comprising from about 1.05 mg to about 3.0 mg of the DHM per mL of the formulation; and/or the Huperzine A is mixed in the solution in an amount to result in the formulation comprising from about 0.26 μg to about 6.6 μg of the Huperzine A per 355 mL of the formulation; and/or the caffeine is mixed in the solution in an amount to result in the formulation comprising from 0.28 mg to about 6.6 mg of the caffeine per mL of the formulation.

According to various aspects of the disclosure, a thirty-sixth aspect can be described as a method for (i) accelerating aldehyde or alcohol metabolism in a subject; or (ii) reducing or reversing one or more negative effects of alcohol on motor or cognition performance in a subject; or (iii) accelerating the reduction of blood-alcohol concentration (BAC) in a subject; or (iv) aiding in the metabolism of an alcohol-containing food or beverage by a subject; or (v) any combination of (i)-(iv); where the method comprises orally administering an ingestible formulation according to any one the first through thirty-fifth aspects to a subject in need thereof.

According to various aspects of the disclosure, a thirty-seventh aspect can be described as a method according to the thirty-sixth aspect, where the subject is a human.

According to various aspects of the disclosure, a thirty-eighth aspect can be described as a use of an ingestible formulation according to any one the first through thirty-fifth aspects for (i) accelerating aldehyde or alcohol metabolism in a subject; or (ii) reducing or reversing one or more negative effects of alcohol on motor or cognition performance in a subject; or (iii) accelerating the reduction of blood-alcohol concentration (BAC) in a subject; or (iv) aiding in the metabolism of an alcohol-containing food or beverage by a subject; or (v) any combination of (i)-(iv).

According to various aspects of the disclosure, a thirty-ninth aspect can be described as a use according to the thirty-eighth aspect, where the subject is a human.

According to various aspects of the disclosure, a fortieth aspect can be described as a method for (i) accelerating aldehyde or alcohol metabolism in a subject; or (ii) reducing or reversing one or more negative effects of alcohol on motor or cognition performance in a subject; or (iii) accelerating the reduction of blood-alcohol concentration (BAC) in a subject; or (iv) aiding in the metabolism of an alcohol-containing food or beverage by a subject; or (v) any combination of (i)-(iv), where the method comprises orally administering about 2 to about 100 g (or about 2 to about 8 g) of fructose and about 40 to about 150 mg methylliberine. In one embodiment, about 4 to about 6 g of fructose (e.g., about 4 g or about 6 fructose) and about 48 to about 150 mg methylliberine (e.g., about 48, about 60, about 100, or about 150 mg methylliberine) are administered. In some instances, the fructose and methylliberine are administered at a weight ratio of about 10:1 to about 500:1, about 25:1 to about 125:1, or about 40:1 to about 100:1 (e.g., at about 25:1, about 27:1, about 40:1, about 50:1, about 100:1, or about 125:1). In one embodiment, the subject is intoxicated and is administered the fructose and methylliberine within one or two hours of intoxication.

According to various aspects of the disclosure, a forty-first aspect can be described as a method according to the fortieth aspect, where the subject is a human subject described herein, such as one having a BAC of at least 0.8% prior to administration.

### BRIEF DESCRIPTION OF THE DRAWINGS

FIG. 1 is a graph showing the percentage of individuals still intoxicated after administration of a Formulation A or Formulation B as described in Example 1.1 over time.

FIG. 2 is a graph showing the blood-alcohol concentration (BAC) over time in intoxicated subjects treated with a Formulation A or Formulation B as described in Example 1.2.

### DETAILED DESCRIPTION

In the context of the present disclosure, it should be understood that comparative or relative phrases related to an effect associated with ingestion of a formulation, such as “accelerates”, “reduces”, “aids”, “helps”, “assists” or “enhances”, or “helps accelerate”, “help reduce”, “helps aid” or “helps enhance” are in comparison to the noted effect without ingestion of the formulation. For example, the phrase “a formulation according to the present disclosure may accelerate aldehyde or alcohol metabolism” should be understood to be equivalent to “the average rate of aldehyde or alcohol metabolism in a subject who ingests the formulation may be greater than the average rate of aldehyde or alcohol metabolism in such a subject when does not ingest the formulation”.

One or more symptoms or acute effects associated with alcohol consumption may be reduced or reversed by metabolizing the ingested alcohol or the aldehydes produced by alcohol metabolism, and such metabolites are eliminated from the body in the course of normal body functions. Such metabolism can occur over time, such as by stopping alcohol consumption and waiting for the body to process the alcohol that is absorbed into the body. According to Mothers Against Drug Driving (MADD), it takes about an average of one hour to process one alcohol drink, such as 1.5 oz of 40% alcohol beverage or 5 oz of 12% alcohol beverage or 12 oz of 5% alcohol beverage (ref: Drunk Driving Facts, Apr. 12, 2022, madd.org).

However, it may be desirable to ingest a composition or formulation that would help accelerate such metabolism. Alternatively or additionally, it may be desirable to ingest a composition or formulation that would reduce or reverse one or more negative effects of alcohol on motor or cognition performance. Physiologically, when one ingests alcohol or alcohol containing substances, they are absorbed, and majority of their metabolism occurs in the liver. The byproducts of alcohol metabolism compromise the nervous system hence inebriation, loss of motor control and compromise of other normal functions of the body and mind, depletion or loss of certain cofactors necessary for alcohol metabolism and could also cause cellular damage. Many of these acute effects are reversed if alcohol and its byproducts are metabolized and eliminated from the body. Hence, one may approach this challenge by reversing the compromise due to inebriation (or help with cognition and mental alertness), help accelerate the normal metabolism rate and elimination rate of alcohol and provide any necessary nutrients/cofactors needed to achieve these tasks.

In some embodiments, the present disclosure provides an ingestible formulation whose consumption may accelerate aldehyde or alcohol metabolism in a subject, help reduce or help reverse one or more negative effects of alcohol on motor or cognition performance in a subject, help accelerate the reduction of blood-alcohol concentration (BAC) or Breathalyzer-alcohol content (BrAC) in a subject, and/or aid in the metabolism of an alcohol-containing food or beverage by a subject.

In another aspect, a formulation according to the present disclosure may be used to help accelerate aldehyde or alcohol metabolism in a subject, help reduce or help reverse one or more negative effects of alcohol on motor or cognition performance in a subject, help accelerate the reduction of blood-alcohol concentration (BAC) or breathalyzer-alcohol concentration (BrAC) in a subject, and/or aid in the metabolism of an alcohol-containing food or beverage by a subject.

In another aspect, the present disclosure provides a method for: help accelerating aldehyde or alcohol metabolism in a subject, help reducing or reversing one or more negative effects of alcohol on motor or cognition performance in a subject, help accelerating the reduction of blood-alcohol concentration (BAC) or breathalyzer-alcohol concentration (BrAC) in a subject, and/or aiding in the metabolism of an alcohol-containing food or beverage by a subject. The method includes orally administering to the subject an ingestible formulation according to the present disclosure.

Ingestible formulations according to various aspects of the disclosure include one or more sugars and methylliberine. In some instances, the one or more sugars is selected from the group consisting of fructose, high fructose corn syrup (HFCS), sucrose (for example, table sugar) and lactose (for example, milk sugar). In some instances, the use fructose is preferred. In some instances, the methylliberine can be used in an isolated form. In some instances, methylliberine may be used in the form of an extract from coffee beans, tea, cola nuts, guarana, cocoa, or yerba mate. In some instances, the methylliberine can be provided in ingestible formulations in a commercially available composition that contains methylliberine. In some instances, the methylliberine can be from a commercial product such as Dynamine® The structure of methylliberine is shown in Formula (I).

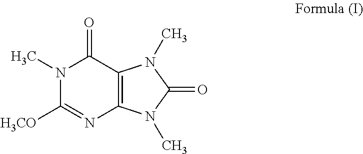 [SMILES: COc1nc2c(c(=O)n1C)n(C)c(=O)n2C]

Ingestible formulations according to the disclosure may include one or more sugars, such as fructose, in a sufficient concentration that a readily ingested amount of the formulations provide enough fructose to help increase alcohol metabolism in a subject. While not wishing to be bound by theory, biochemically, each fructose molecule in the liver generates one molecule of NAD+ when fructose is metabolized, and NAD+ in turn is required for the metabolism of alcohol. Thus, fructose regenerates NAD+ in the liver, thus helping replenish NAD+ in the liver for the metabolism of alcohol. (Berry et al. “Ethanol oxidation by isolated rat-liver cells. Stimulatory effects of fructose.” Eur. J. Biochem., 1978 Aug. 15, 89 (1), pp. 237-41).

Ingestible formulations according to the disclosure may include methylliberine in a sufficient concentration that a readily ingested amount of the formulations provide enough methylliberine to neurologically stimulate a subject ingesting such a formulation. In some instances, the amount of methylliberine in an ingestible formulation is limited by the amount allowed by its own toxicity limits. In other instances, the amount of methylliberine in an ingestible formulation may be limited by the amount allowed by a regulatory agency such as US FDA.

In some instances, ingestible formulations according to the disclosure may include fructose, as the one or more sugars, and methylliberine. In some instances, ingestible formulations according to the disclosure include about 1 mg to about 150 mg of methylliberine, and about 2 grams to about 200 grams of fructose. In some instances, ingestible formulations according to the disclosure include fructose and methylliberine at a weight ratio of about 10:1 to about 500:1, about 25:1 to about 125:1, or about 40:1 to about 100:1 (e.g., at about 25:1, about 27:1, about 40:1, about 50:1, about 100:1, or about 125:1).

In some instances, ingestible formulations according to the disclosure may additionally include dihydromyricetin (DHM) and/or Huperzine A. When an ingestible formulation according to the disclosure includes DHM, the ingestible formulation may include about 50 mg to about 1000 mg of the DHM. When an ingestible formulation according to the disclosure includes Huperzine A, the ingestible formulation may include about 0.1 μg to about 200 μg of the Huperzine A.

In some instances, DHM (which may also be known as ampelopsin) can be used in the form of an extract from kale, oranges, or vine tea. The structure of DHM is shown in Formula (II) and the structure.

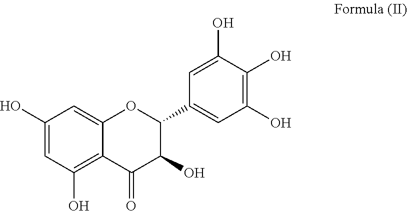 [SMILES: O=C1c2c(O)cc(O)cc2O[C@H](c2cc(O)c(O)c(O)c2)[C@H]1O]

An ingestible formulation according to various aspects of the disclosure may include DHM in a sufficient concentration that a readily ingested amount of the formulation provides enough DHM to reduce alcohol intoxication and/or increase alcohol metabolism in the subject (Silva et al., “Dihydromyricetin Protects the Liver via Changes in Lipid Metabolism and Enhanced Ethanol Metabolism”, Alcohol Clin Exp Res., 2020, 44 (5), pp. 1046-1060; (b) Shen et al., “Dihydromyricetin as a novel anti-alcohol intoxication medication”, J. Neurosci., 2012; 32, pp. 390-401).

Huperzine A can be extracted from Huperzia serrata. The structure of Huperzine A is shown in Formula (III).

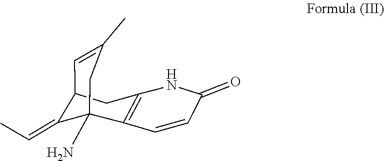 [SMILES: C/C=C1\C2C=C(C)CC1(N)c1ccc(=O)[nH]c1C2]

An ingestible formulation according to various aspects of the disclosure may include Huperzine A in a sufficient concentration that a readily ingested amount of the formulation provides enough Huperzine A to support and/or enhance the cognitive abilities of the subject (Wang et al., “Huperzine A improves cognitive deficits caused by chronic cerebral hypoperfusion in rats”, Eur. J. Pharmacol., 2000, 398 (1), pp. 65-72; Xu et al., “Huperzine-A in capsules and tablets for treating patients with Alzheimer disease”, Acta Pharmacologica Sinica, 1999, 20 (6), pp. 486-490; Sun et al., “Huperzine-A capsules enhance memory and learning performance in 34 pairs of matched adolescent students”, Acta Pharmacologica Sinica, 1999, 20 (7), pp. 601-603.)

In some instances, ingestible formulations according to the disclosure may further include one or more of: caffeine, N-acetyl tyrosine (NAT), methylcobalamin, pyridoxal-5-phosphate, pyridoxine or its salt forms such as HCl, thiamine, riboflavin, niacin, nicotinamide, quercetin, L-phenylalanine, calcium citrate, magnesium citrate, sodium citrate, and potassium chloride or monopotassium phosphate. In some instances, one or more of the above compounds may be used in their derivative forms such as isoquercitrin-CD Complex (70% γ-Cyclodextrin, 30% quercetin glycoside complex, or Isoquercitrin, rhamnose). Derivative forms of the above compounds may be useful in instances where they produce the original compound in vivo, such as the in vivo production of quercetin from a derivative form thereof.

In some instances, ingestible formulations according to the disclosure may include caffeine. In some instances, the caffeine can be provided by including appropriate amounts of extracts of green tea extract, organic green tea extract, coffee beans. When an ingestible formulation according to the disclosure includes caffeine or in the form of extracts of green tea or coffee beans, the ingestible formulation may include about 100 mg to about 400 mg of caffeine.

Ingestible formulations according to various aspects of the disclosure may further include one or more nutritional supports or electrolytes or cofactors selected from: N-acetyl tyrosine (NAT), methylcobalamin, pyridoxal 5-phosphate, pyridoxine HCl, thiamine, riboflavin, niacin, nicotinamide, quercetin, L-phenylalanine, calcium citrate, magnesium citrate, sodium citrate, and potassium chloride or monopotassium phosphate. The formulation may include the one or more nutritional supports or electrolytes or cofactors in a sufficient concentration that a readily ingested amount of the formulation provides enough of the one or more nutritional supports or electrolytes or cofactors to: replenish diminished concentrations of or supply added quantities of the respective nutritional support or electrolyte or cofactor necessary to help accelerate the metabolism of alcohol, and/or help enhance cognition and/or help recover from the insult caused by alcohol and its byproducts.

In some instances, ingestible formulations according to the disclosure, can include about 0.10 mg to about 5.0 mg of methylcobalamin or vitamin B12, but can be as low as 0.1 mcg; about 0.05 mg to about 80 mg of pyridoxal 5-phosphate, pyridoxine HCl, or vitamin B6 or a combination of pyridoxal 5-phosphate and pyridoxine HCl; about 0.5 mg to about 3 mg of thiamine; about 2.5 mg to about 10 mg of riboflavin; about 5 mg to about 20 mg of niacin, nicotinamide, or a combination of niacin and nicotinamide, or may be up to 500 mg; about 250 mg to about 1,200 mg of quercetin, or a molar equivalent amount of a derivative of quercetin; about 250 mg to about 1,000 mg of L-phenylalanine; about 100 mg to about 400 mg of calcium citrate; about 100 mg to about 400 mg of magnesium citrate; about 100 mg to about 400 mg of sodium citrate; and about 100 mg to about 400 mg of potassium chloride or monopotassium phosphate.

In some instances, ingestible formulations according to various aspects of the disclosure may additionally include an extract of milk thistle. Silymarin, the active ingredient in milk thistle, acts as a free radical scavenger and modulates enzymes associated with the development of cellular damage, fibrosis and cirrhosis (Kwon et al., “Alterations in sulfur amino acid metabolism in mice treated with silymarin: a novel mechanism of its action involved in enhancement of the antioxidant defense in liver”, Planta medica, 2013, 79 (12), pp. 997-1002). This compound also is shown to possess anti-inflammatory properties and inhibits the production of various cytokines (Li et al., “Identification of novel mechanisms of silymarin on the carbon tetrachloride-induced liver fibrosis in mice by nuclear factor-kappaB bioluminescent imaging-guided transcriptomic analysis”, Food Chem. Toxicol., 2012, 50 (5), pp. 1568-1575; Gharagozloo et al., “Silymarin suppress CD4+ T cell activation and proliferation: effects on NF-kappaB activity and IL-2 production”, Pharmacol. Res., 2010, 61 (5), pp. 405-409; Trappoliere et al., “Silybin, a component of sylimarin, exerts anti-inflammatory and anti-fibrogenic effects on human hepatic stellate cells”, J. Hepatol. 2009, 50 (6), pp. 1102-1111). It also reduces the cellular uptake of xenobiotics—a result of chemical exposure, by blocking organic ion uptake transporters in hepatocytes (Faulstich et al., “Silybin inhibition of amatoxin uptake in the perfused rat liver”, Arzneim. Forsch., 1980, 30 (3), pp. 452-454.).

In some instances, the milk thistle extract, containing silymarin, may be present in an ingestible formulation according to the disclosure in a sufficient concentration that a readily ingested amount of the formulation provides enough milk thistle extract to act as a liver protectant. When present, ingestible formulations according to the disclosure may include about 30 mg to about 600 mg of silymarin or an equivalent amount of milk thistle extract.

In some instances, ingestible formulations according to various aspects of the disclosure may additionally include a preservative, such as potassium sorbate, sodium benzoate, citric acid, or any combination thereof; and/or a flavouring agent. In some instances, one or more preservatives may not be included, but methods such as pasteurization may be used after the formulation is prepared.

In some instances, ingestible formulations according to various aspects of the disclosure may additionally include a pH adjustment component and water. In some instances, water may be substituted partially or fully with an appropriate juice such as sweet lime juice, pear juice, tomato juice. The pH of a liquid formulation may be from about pH 3 to about pH 5.5, such as a pH of about 4.

Ingestible formulations according various aspects of the disclosure may be formulated in many ingestible dosage forms, for example as a beverage. In other forms, one may design a powder or other forms of solid dosage forms which can be dissolved or dispersed in water or juices in order to ingest the formulation. A readily ingested amount of an ingestible formulation according the disclosure depends on the format of said formulation. A readily ingested amount of a beverage may be, for example, the volume of liquid that an adult subject could comfortably ingest immediately, or within 10-20 minutes. For example, a unit or individual dosage of a beverage formulation may be from about 2 to about 12 or 16 fluid oz (about 60 mL to about 355 mL or 473 mL) in volume per serving size. In contrast, a readily ingested amount of a powder, a solid dosage form, gel or paste may be, for example, the volume of such ingestible formulations that adult subject could comfortably ingest within 5 minutes.

For example, a unit dosage of a gel or paste formulation may be from about 5 grams to about 75 grams. One may also use a powder formulation or a solid formulation from about 5 grams to about 50 grams, then dissolved or dispersed in water or juices or other ingestible fluids for ingestion.

In some examples, alcohol concentration may be measured by using a breathalyzer and collecting a suitable volume of blood from the subjects, at various times, and quantifying the amount of alcohol in the corresponding breath or blood samples. If alcohol metabolism is accelerated, one will observe a reduction in the concentration of alcohol in the breath and/or blood samples over time.

An ingestible formulation according to the present disclosure may be used in a method to accelerate aldehyde or alcohol metabolism in a subject; reduce or reverse one or more negative effects of alcohol on motor or cognition performance in a subject; accelerate the reduction of blood-alcohol concentration (BAC) or breathalyzer alcohol concentration (BrAC) in a subject; aid in the metabolism of an alcohol-containing food or beverage by a subject; or any combination thereof. The ingestible formulation is orally administered to the subject. As noted above, comparative or relative phrases related to an effect associated with ingestion of a formulation, such as “accelerates”, “reduces”, “aids”, “helps”, “assists” or “enhances”, are in comparison to the noted effect without ingestion of the formulation. The subject may be a mammal, such as a human, and preferably an adult human. Alcohol concentration may be measured by using a breathalyzer and collecting a suitable volume of blood from the subjects, at various times, and quantifying the amount of alcohol in the corresponding breath or blood samples. If alcohol metabolism is accelerated, one will observe a reduction in the concentration of alcohol in the breath and/or blood samples over time.

In accordance with various aspects of the disclosure, methods of rapidly reducing the blood alcohol concentration in an intoxicated human subject comprising orally administering to the subject an ingestible formulation according to the disclosure, wherein the blood alcohol concentration of the subject is reduced to no more than 0.08%, 0.07%, 0.06%, 0.05%, 0.04%, 0.03%, or 0.02%, within 15 minutes, 20 minutes, 30 minutes, 45 minutes, one hour, two hours, three hours, or four hours, of administration of the ingestible formulation. In some instances, the intoxicated human subject, prior to oral administration of the ingestible formulation, has a blood alcohol concentration of at least 0.09%, 0.10%, 0.11%, 0.12%, 0.13%, 0.14%, 0.15%, 0.20%, 0.25%, 0.30%, 0.35%, or 0.40%.

In accordance with various aspects of the disclosure, methods of rapidly reducing the blood alcohol concentration in an intoxicated human subject comprising orally administering to the subject about 2 to about 100 g (or about 2 to about 8 g) of fructose and about 40 to about 150 mg methylliberine, wherein the blood alcohol concentration of the subject is reduced to no more than 0.08%, 0.07%, 0.06%, 0.05%, 0.04%, 0.03%, or 0.02%, within 15 minutes, 20 minutes, 30 minutes, 45 minutes, one hour, two hours, three hours, or four hours, of administration of the ingestible formulation. In some instances, the intoxicated human subject, prior to oral administration of the ingestible formulation, has a blood alcohol concentration of at least 0.09%, 0.10%, 0.11%, 0.12%, 0.13%, 0.14%, 0.15%, 0.20%, 0.25%, 0.30%, 0.35%, or 0.40%. In one embodiment, about 4 to about 6 g of fructose (e.g., about 4 g or about 6 fructose) and about 48 to about 150 mg methylliberine (e.g., about 48, about 60, about 100, or about 150 mg methylliberine) are administered. In some instances, the fructose and methylliberine are administered at a weight ratio of about 10:1 to about 500:1, about 25:1 to about 125:1, or about 40:1 to about 100:1 (e.g., at about 25:1, about 27:1, about 40:1, about 50:1, about 100:1, or about 125:1). In one embodiment, the subject is intoxicated and is administered the fructose and methylliberine within one or two hours of intoxication.

An ingestible formulation according to the present disclosure may be prepared in a process that includes: mixing methylliberine and fructose in a solution that includes water in amounts to result in a formulation that includes from about 0.05 mg to about 4.0 mg, alternatively about 0.1 mg to about 3.3 mg, alternatively about 0.12 mg to about 3.3 mg, and alternatively about 0.2 mg to about 0.8 mg of the methylliberine per mL of the formulation; and from about 1 mg to about 200 mg, alternatively about 5 mg to about 150 mg, alternatively about 10 mg to about 125 mg, and alternatively 12 mg to about 32 mg of the fructose per mL of the formulation.

The process may additionally include any combination of the following: (a) mixing dihydromyricetin (DHM) in the solution, such as in an amount to result in the formulation comprising from about 0.8 mg to about 3.4 mg, and alternatively, alternatively about 1.0 mg to about 3.2 mg, and alternatively about 1.05 mg to about 3.0 mg of DHM per mL of the formulation; (b) mixing Huperzine A in the solution, such as in an amount to result in the formulation comprising from about 0.2 μg to about 7.0 μg, alternatively 0.26 μg to about 6.6 μg, and alternatively 0.3 μg to about 1.6 μg of Huperzine A per 355 mL of the formulation; (c) mixing caffeine (in the form of, for example, extract of organic green tea) in the solution, such as in an amount to result in the formulation comprising from 0.1 mg to about 1 mg, alternatively about 0.2 mg to about 0.75 mg, and alternatively about 0.28 mg to about 0.66 mg of caffeine per mL of the formulation.

Table 1 outlines ingestible formulations according to various aspects of the disclosure and amounts (ranges) of ingredients that may be used per 12 oz (355 mL) serving, in water or an appropriate juice, in carbonated or uncarbonated form.

TABLE 1

| | |
|---|---|
| | Amount (range) per 12 oz (355 mL) |
| | serving, in water or an appropriate |
| Ingredient | juice, in carbonated or uncarbonated form |

| | | |
|---|---|---|
| Fructose | 2-100 | g |
| B12 | 10-400 | mcg |
| (methylcobalamin) | | |
| B6 (P5P) | 1-30 | mg |
| B1 (thiamine) | 0.5-5 | mg |
| B2 (Riboflavin) | 0.5-15 | mg |
| Magnesium Citrate | 10-400 | mg |
| Sodium citrate | 10-400 | mg |
| Potassium chloride | 10-400 | mg |
| or Monopotassium | | |
| phosphate | | |
| Potassium sorbate | 0.005%-0.1% | w/v |
| Sodium benzoate | 0.01%-0.1% | w/v |
| Citric acid | 0.05%-0.5% | w/v |
| Lecithin | 0.05%-1% | w/v |
| Sucralose | 0.02%-0.1% | w/v |

| | |
|---|---|
| DHM (in vine | 50 mg-1000 mg |
| tea extract) | |
| Methylliberine | 1 mg-200 mg |
| (or products that | |
| deliver | |
| methylliberine) | |
| Quercetin (or | 250 mg-1,200 mg |
| products that | |
| deliver quercetin) | |
| Milk thistle | 30 mg-600 mg |
| L-phenylalanine | 250 mg-1,000 mg |
| Huperzine A | 0.1 mcg-200 mcg |
| Organic green | 0 mg-400 mg |
| tea extract | |
| (caffeine) | |
| Niacin | 0 mg-100 mg |
| NAT | 0 mg-1,500 mg |

Table 2 outlines three additional exemplary formulations (A-C) according to the disclosure and amounts of ingredients that may be used per 12 oz (355 mL) serving, in water or an appropriate juice, in carbonated or uncarbonated form.

TABLE 2

| | Formulation-Amount per 12 oz (355 mL) | | | | | |
|---|---|---|---|---|---|---|
| | serving, in water or an appropriate | | | | | |
| | juice, in carbonated or uncarbonated form | | | | | |
| Ingredient | A | | B | | C | |
| Fructose | 6 | g | 6 | g | 6 | g |
| B12 | 178 | mcg | 178 | mcg | 178 | mcg |
| (methylcobalamin) | | | | | | |
| B6 (P5P) | 11 | mg | 11 | mg | 11 | mg |
| B1 (thiamine) | 1.4 | mg | 1.4 | mg | 1.4 | mg |
| B2 (Riboflavin) | 5 | mg | 5 | mg | 5 | mg |
| Magnesium Citrate | 200 | mg | 200 | mg | 200 | mg |
| Sodium citrate | 200 | mg | 200 | mg | 200 | mg |
| Potassium chloride | 200 | mg | 200 | mg | 200 | mg |
| or monopotassium | | | | | | |
| phosphate | | | | | | |
| Potassium sorbate | 0.03% | w/v | 0.03% | w/v | 0.03% | w/v |
| Sodium benzoate | 0.03% | w/v | 0.03% | w/v | 0.03% | w/v |
| Citric acid | 0.15% | w/v | 0.15% | w/v | 0.15% | w/v |
| Lecithin | 0.25% | w/v | 0.25% | w/v | 0.25% | w/v |
| Sucralose | 0.02% | w/v | 0.02% | w/v | 0.02% | w/v |
| DHM (from Vine | 150 | mg | 600 | mg | 400 | mg |
| tea extract) | | | | | | |
| Methylliberine | 48 | mg | 60 | mg | 48 | mg |
| Quercetin | 500 mg (or an | | 500 mg (or an | | 500 mg (or an | |
| | equivalent | | equivalent | | equivalent | |
| | amount of a | | amount of a | | amount of a | |
| | product | | product | | product | |
| | providing | | providing | | providing | |
| | quercetin) | | quercetin) | | quercetin) | |
| Milk thistle | 60 | mg | 60 | mg | 180 | mg |
| L-phenylalanine | 500 | mg | 500 | mg | 500 | mg |
| Huperzine A | 1.25 | mcg | 1.50 | mcg | 1.50 | mcg |

| | | | | | |
|---|---|---|---|---|---|
| Organic green tea | 150 | mg | 0 | 200 | mg |
| extract (caffeine) | | | | | |
| Niacin | 10 | mg | 0 | 10 | mg |

| | | | | |
|---|---|---|---|---|
| NAT | 0 | 0 | 500 | mg |

Table 3 outlines yet another exemplary ingestible formulation according to various aspects of the disclosure and amounts (ranges) of ingredients that may be used per 12 oz (355 mL) serving, in water or an appropriate juice, in carbonated or uncarbonated form.

TABLE 3

| | Amount (range) per 12 oz (355 mL) | |
|---|---|---|
| | serving, in water or an appropriate | |
| Ingredient | juice, in carbonated or uncarbonated form | |
| Fructose | 6 | g |
| Methylcobalamin | 178 | mcg |
| Pyridoxine HCl | 10 | mg |
| Thiamine hydrochloride | 1.4 | mg |
| Riboflavin | 5 | mg |
| Magnesium Citrate | 200 | mg |
| Sodium citrate | 200 | mg |
| Potassium chloride | 10-400 | mg |
| monopotassium phosphate | 181 | mg |
| DHM (from Vine | 600 | mg |
| Tea Extract) | | |
| Methylliberine | 60 | mg |
| Isoquercitrin complex | 181 | mg |
| Milk Thistle Extract | 60 | mg |
| L-phenylalanine | 500 | mg |
| Huperzia Serrata Extract | 150 | mcg |
| (1% Huperzine A) | | |
| Green Tea Extract | 200 | mg |
| (98% Caffeine) | | |
| Niacin | 10 | mg |
| Citric acid (Optional) | 0.2-0.5% | w/v |
| Xanthan gum (Optional) | 0.04-0.06% | w/w |
| Sucralose (Optional) | 0.01-0.03% | w/w |

In some examples, an ingredient noted herein may be replaced with functionally equivalent compound. The term “functionally equivalent” refers herein to a compound which retains the biological activity of another compound. The functionally equivalent compounds need not exhibit identical activity to reference compound. Put another way, “functionally equivalent” refers to a compound that has the same or substantially the same function as another compound.

In some examples, fructose may be functionally equivalent to high fructose corn syrup (HFCS), sucrose (table sugar), lactose (milk sugar), etc.

In some examples, B12 may be functionally equivalent to Cyanocobalamin, Hydroxocobalamin, Adenosylcobalamin, Cobamamide, and Methylcobalamin.

In some examples, B1 may be functionally equivalent to thiamine mononitrate, thiamine hydrochloride, allithiamine, benfotiamine, and sulbutiamine.

In some examples, B2 may be functionally equivalent to flavin mononucleotide (FMN), flavin adenine dinucleotide (FAD), lumiflavin, riboflavin 5′-phosphate, and riboflavin tetraacetate.

In some examples, magnesium citrate may be functionally equivalent to magnesium glycinate, magnesium oxide, magnesium sulfate, magnesium malate, and magnesium chloride.

In some examples, sodium citrate may be functionally equivalent to potassium citrate, calcium citrate, magnesium citrate, citric acid, and sodium bicarbonate.

In some examples, potassium chloride may be functionally equivalent to monopotassium phosphate, potassium citrate, potassium gluconate, potassium bicarbonate, potassium aspartate, and potassium iodide.

In some examples, potassium sorbate may be functionally equivalent to sodium benzoate, calcium propionate, sodium propionate, potassium benzoate, monopotassium phosphate, and sorbic acid.

In some examples, sodium benzoate may be functionally equivalent to potassium benzoate, benzoic acid, calcium benzoate, potassium sorbate, monopotassium phosphate, and sodium propionate.

In some examples, citric acid may be functionally equivalent to tartaric acid, malic acid, acetic acid, lactic acid, and ascorbic acid.

In some examples, lecithin may be functionally equivalent to phosphatidylcholine, phosphatidylserine, sphingomyelin, glycosphingolipids, and polyunsaturated fatty acids (PUFAs).

In some examples, sucralose may be functionally equivalent to aspartame, saccharin, stevia, erythritol, and xylitol.

In some examples, vine tea extract may be functionally equivalent to resveratrol, quercetin, naringenin, baicalin, and curcumin.

In some examples, methylliberine may be functionally equivalent to caffeine, theobromine, theacrine, synephrine, and hordenine.

In some examples, Iso-Q may be functionally equivalent to quercetin, kaempferol, rutin, hesperidin, and luteolin.

In some examples, milk thistle may be functionally equivalent to silibinin, N-acetylcysteine (NAC), curcumin, resveratrol, and schisandra.

In some examples, L-phenylalanine may be functionally equivalent to L-tyrosine, DL-phenylalanine, phenylethylamine (PEA), L-3,4-dihydroxyphenylalanine (L-DOPA), or 5-hydroxytryptophan (5-HTP).

In some examples, Huperzine A may be functionally equivalent to galantamine, bacopa monnieri, Ginkgo biloba, phosphatidylserine, and caffeine.

In some examples, organic green tea extract (caffeine) may be functionally equivalent to epigallocatechin gallate (EGCG), L-theanine, theobromine, chlorogenic acid, and catechins.

In some examples, Niacin may be functionally equivalent to nicotinamide, nicotinamide riboside, inositol hexanicotinate, tryptophan, and pantothenic acid.

### EXAMPLES

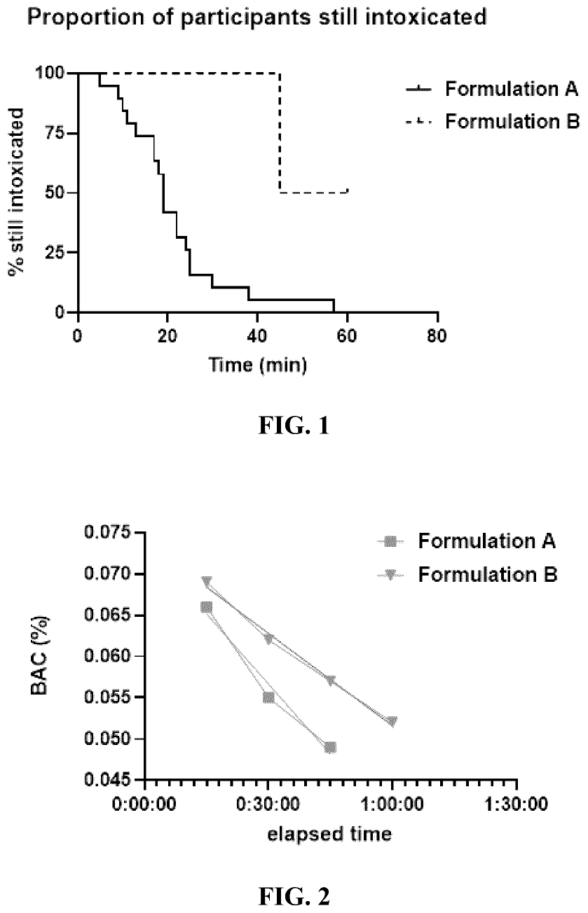

Example 1.1. In a focus group study, two types of formulations (hereafter referred to as Formulation Type A and Type B) were evaluated their effects on the inebriation and the rate of alcohol metabolism. Each Formulation Type A contained 48, 60, 100 or 150 mg methylliberine and 4 or 6 g fructose, Formulation Type B did not contain methylliberine and fructose. The group consuming Formulation A exhibited faster recovery from inebriation from intoxication (solid line in FIG. 1). In the group that consumed Formulation B that did not contain methylliberine and fructose, participants exhibited inebriation due to alcohol intoxication for an extended period of time, and only 50% recovered after 40 minutes (dotted line in FIG. 1). This study supports the effect of methylliberine and fructose on inebriation. Inebriation was self-reported using a visual analog scale (VAS) from 0-10, where inebriation is defined as having a value >2 on scale. For the data of FIG. 1, there were 19 participants in the group receiving a Type A formulation and four participants in the group receiving Formulation Type B.

Example 1.2. In a separate study, using one person as a control, both Type A Formulations and Formulation Type B were tested on two separate occasions and breath alcohol concentrations (BAC) were measured to see if there is any difference between Type A Formulations and Formulation Type B. Subjects of this study were found to have an average BAC of 0.079% (range of 0.044-0.157%) prior to administration of a formulation. All subjects of the focus groups were 20 years old or older. The formulations were administered to the subjects orally over a five minute period. As illustrated in FIG. 2, BAC elimination slopes were obtained using linear regression from BAC measurements after taking the formulations. Formulation Type B showed a lower rate of decrease in BAC, in comparison to the Type A formulations. Specifically, the BAC elimination slope was −0.034%/hr with Type A Formulations and −0.022%/hr with Formulation Type B. This suggests that alcohol elimination is enhanced with Type A formulations, with fructose and methylliberine, compared with Formulation Type B without fructose and methylliberine. Fructose provides supplemental NAD+ through its metabolism in the liver, and NAD+ is an important cofactor for the metabolism of ethanol.

In the preceding description, for purposes of explanation, numerous details are set forth in order to provide a thorough understanding of the examples. However, it will be apparent to one skilled in the art that these specific details are not required. Accordingly, what has been described is merely illustrative of the application of the described examples and numerous modifications and variations are possible in light of the above teachings.

Since the above description provides examples, it will be appreciated that modifications and variations can be effected to the particular examples by those of skill in the art. Accordingly, the scope of the claims should not be limited by the particular examples set forth herein, but should be construed in a manner consistent with the specification as a whole.

1. An ingestible formulation comprising:
one or more sugars selected from the group consisting of fructose, high fructose corn syrup (HFCS), sucrose and lactose; and
methylliberine,
optionally wherein coffee beans, tea, cola nuts, guarana, cocoa, and/or yerba mate provides the methylliberine.

2. The ingestible formulation of claim 1, wherein the one or more sugars is fructose.

3. The ingestible formulation of claim 1, comprising:
about 1 mg to about 150 mg of the methylliberine, and
about 2 g to about 100 g of the fructose.

4. The ingestible formulation of claim 1, further comprising dihydromyricetin (DHM).

5. The ingestible formulation of claim 4, wherein the formulation comprises:
about 1 mg to about 150 mg of methylliberine,
about 2 grams to about 100 grams of fructose, and
about 50 mg to about 1000 mg of DHM.

6. The ingestible formulation of claim 4, further comprising Huperzine A.

7. The ingestible formulation of claim 6, wherein the formulation comprises:
about 1 mg to about 150 mg of methylliberine,
about 2 grams to about 100 grams of fructose, and
about 50 mg to about 1000 mg of DHM and
about 0.1 μg to about 200 μg of Huperzine A.

8-9. (canceled)

10. The ingestible formulation of claim 1, wherein the formulation comprises one or more of:
about 5 mg to about 100 mg of niacin, nicotinamide, or a combination of niacin and nicotinamide;
about 50 mg to about 1,200 mg of quercetin;
about 250 mg to about 1,000 mg of L-phenylalanine,
about 0.1 mg to about 5.0 mg of methylcobalamin;
about 5 mg to about 100 mg of pyridoxal 5-phosphate (PSP), pyridoxine HCl, or a combination of pyridoxal 5-phosphate and pyridoxine HCl;
about 0.5 mg to about 3 mg of thiamine;
about 2.5 mg to about 10 mg of riboflavin;
about 100 mg to about 400 mg of calcium citrate,
about 100 mg to about 400 mg of magnesium citrate,
about 100 mg to about 400 mg of sodium citrate, and
about 100 mg to about 400 mg of potassium chloride or monopotassium phosphate.

11. The ingestible formulation of claim 1, further comprising:
an extract of milk thistle; or
silymarin.

12. The ingestible formulation of claim 11, wherein the formulation comprises about 30 mg to about 600 mg of the extract of milk thistle or silymarin.

13-14. (canceled)

15. The ingestible formulation of claim 1, wherein the formulation comprises fructose, B12 (methylcobalamin), B6 (PSP), B1 (thiamine), B2 (riboflavin), magnesium citrate, sodium citrate, potassium chloride (or monopotassium phosphate), potassium sorbate, sodium benzoate, citric acid, lecithin, sucralose, vine tea extract, methylliberine (or a methylliberine-containing compound or composition), quercetin (or a quercetin-containing compound or composition), milk thistle or an extract thereof, L-phenylalanine, Huperzia serrata (Huperzine A), optionally organic green tea extract, and optionally Niacin or NAT.

16. The ingestible formulation of claim 15, wherein the formulation comprises:
2-100 g fructose;
10-400 mcg B12 (methylcobalamin);
1-30 mg B6 (P5P);
0.5-5 mg B1 (thiamine);
0.5-15 mg B2 (riboflavin);
10-400 mg magnesium citrate;
10-400 mg sodium citrate;
10-400 mg potassium chloride or monopotassium phosphate;
0.005%-0.1% w/v potassium sorbate;
0.01%-0.1% w/v sodium benzoate;
0.05%-0.5% w/v citric acid;
0.05%-1% w/v lecithin;
0.02%-0.1% w/v sucralose;
50-1000 mg of DHM provided by vine tea extract;
1-200 mg methylliberine (or a methylliberine-containing compound or composition containing 1-200 mg methylliberine);
250-1,200 mg quercetin (or a quercetin-containing compound or composition containing 250-1,200 mg quercetin);
30-600 mg of milk thistle or the extract thereof;
250-1,000 mg L-phenylalanine;
0.1-200 mcg Huperzine A;
0-400 mg organic green tea extract;
0-100 mg Niacin; and
0-1,500 mg NAT.

17. The ingestible formulation of claim 15, wherein the formulation comprises:
6 g fructose;
178 mcg B12 (methylcobalamin);
11 mg B6 (P5P);
1.4 mg B1 (thiamine);
5 mg B2 (riboflavin);
200 mg magnesium citrate;
200 mg sodium citrate;
200 mg potassium chloride or monopotassium phosphate;
0.03% w/v potassium sorbate;
0.03% w/v sodium benzoate;
0.15% w/v citric acid;
0.25% w/v lecithin;
0.02% w/v sucralose;
150 mg of DHM provided by vine tea extract;
48 mg methylliberine (or a methylliberine-containing compound or composition containing 48 mg methylliberine),
500 mg quercetin (or a quercetin-containing compound or composition containing 500 mg quercetin),
60 mg milk thistle or the extract thereof;
500 mg L-phenylalanine;
125 mcg Huperzia serrata extract (comprising 1 w/w % Huperzine A));
150 mg organic green tea extract; and
10 mg niacin.

18. The ingestible formulation of claim 15, wherein the formulation comprises:
6 g fructose;
178 mcg B12 (methylcobalamin);
11 mg B6 (P5P);
1.4 mg B1 (thiamine);
5 mg B2 (riboflavin);
200 mg magnesium citrate;
200 mg sodium citrate;
200 mg potassium chloride or monopotassium phosphate;
0.03% w/v potassium sorbate;
0.03% w/v sodium benzoate;
0.15% w/v citric acid;
0.25% w/v lecithin;
0.02% w/v sucralose;
600 mg DHM provided by vine tea extract;
60 mg methylliberine (or a methylliberine-containing compound or composition containing 60 mg methylliberine);
500 mg quercetin (or a quercetin-containing compound or composition containing 500 mg quercetin);
60 mg milk thistle or the extract thereof;
500 mg L-phenylalanine; and
150 mcg Huperzia serrata extract (comprising 1 w/w % Huperzine A).

19. The ingestible formulation of claim 15, wherein the formulation comprises:
6 g fructose;
178 mcg B12 (methylcobalamin);
11 mg B6 (P5P);
1.4 mg B1 (thiamine);
5 mg B2 (riboflavin);
200 mg magnesium citrate;
200 mg sodium citrate;
200 mg potassium chloride or monopotassium phosphate;
0.03% w/v potassium sorbate;
0.03% w/v sodium benzoate;
0.15% w/v citric acid;
0.25% w/v lecithin;
0.02% w/v sucralose;
400 mg DHM provided by vine tea extract;
48 mg methylliberine (or a methylliberine-containing compound or composition containing 48 mg methylliberine),
500 mg quercetin (or a quercetin-containing compound or composition or precursor containing 500 mg quercetin),
180 mg milk thistle or the extract thereof;
500 mg L-phenylalanine;
150 mcg Huperzia serrata extract (comprising 1 w/w % Huperzine AHuperzine A);
200 mg organic green tea extract;
10 mg niacin; and
500 mg NAT.

20. The ingestible formulation of claim 1, wherein the formulation comprises fructose, methylcobalamin, pyridoxine hydrochloride, thiamine hydrochloride, riboflavin, niacin, magnesium citrate, sodium citrate, monopotassium phosphate, vine tea extract, methylliberine (or a methylliberine-containing compound or composition), quercetin (or a quercetin-containing compound or composition, or a quercetin precursor), milk thistle or an extract thereof, L-phenylalanine, Huperzia serrata (comprising of 1 w/w % Huperzine A), green tea extract.

21. The ingestible formulation of claim 20, wherein the formulation comprises:
6 g fructose;
178 mcg methylcobalamin;
10 mg pyridoxine hydrochloride;
1.4 mg thiamine hydrochloride;
5 mg riboflavin;
10 mg niacin;
200 mg magnesium citrate;
200 mg sodium citrate;
200 mg monopotassium phosphate;
600 mg DHM provided by vine tea extract;
60 mg methylliberine (or a methylliberine-containing compound or composition);
181 mg quercetin (or a quercetin-containing compound or composition, or a quercetin precursor),
60 mg milk thistle or an extract thereof;
500 mg L-phenylalanine;
150 mcg Huperzia serrata (comprising 1 w/w % Huperzine A); and
200 mg green tea extract.

22-26. (canceled)

27. An ingestible formulation prepared by a process comprising:
mixing methylliberine and fructose in a solution comprising water in amounts to result in a formulation that comprises:
from about 0.12 mg to about 3.3 mg of the methylliberine per mL of the formulation; and
from about 11 mg to about 125 mg of the fructose per mL of the formulation.

28-30. (canceled)

31. A method for:
(i) accelerating aldehyde or alcohol metabolism in a subject; or
(ii) reducing or reversing one or more negative effects of alcohol on motor or cognition performance in a subject; or
(iii) accelerating the reduction of blood-alcohol concentration (BAC) in a subject; or
(iv) aiding in the metabolism of an alcohol-containing food or beverage by a subject; or
(v) any combination of (i)-(iv);
the method comprising:
orally administering an ingestible formulation according to claim 3 to a subject in need thereof.

32-36. (canceled)

37. A method of rapidly reducing the blood alcohol concentration in an intoxicated human subject comprising orally administering to the subject an effective amount of the ingestible formulation of claim 1, wherein the blood alcohol concentration of the subject is reduced to no more than 0.08% within 30 minutes of administration of the ingestible formulation.

38-40. (canceled)
